(19) United States
(12) Patent Application Publication
(10) Pub. No.: US 2016/0271251 A1
(43) Pub. Date: Sep. 22, 2016

(54) RADIOLUMINESCENT COMPOUND FOR RADIOTHERAPY AND DEEP PHOTODYNAMIC THERAPY AND DEVICE FOR DEEP PHOTODYNAMIC THERAPY

(71) Applicants: SYNCHROTRON SOLEIL, St Aubin (FR); INSTITUT NATIONAL DE LA RECHERCHE AGRONOMIQUE - INRA, Paris (FR)

(72) Inventors: Slavka KASCAKOVA, Paris (FR); Matthieu REFREGIERS, Paris (FR); Alexandre GIULIANI, Paris (FR)

(73) Assignee: SYNCHROTRON SOLEIL, St Aubin (FR)

(21) Appl. No.: 15/030,895

(22) PCT Filed: Sep. 30, 2014
(86) PCT No.: PCT/FR2014/052478

§ 371 Date: Apr. 21, 2016

(30) Foreign Application Priority Data
Oct. 23, 2013 (FR) 13 60353

(51) Int. Cl. A61K 41/00; A61N 5/06; A61N 5/10

(52) U.S. Cl. CPC A61K 41/0057; A61K 41/0071; A61N 5/10; A61N 5/062; A61N 2005/1098

(57) ABSTRACT

A radioluminescent compound for radiotherapy and deep photodynamic therapy (Deep PDT), the radioluminescent compound including a molecular conjugate made up of a radioluminescent molecule and one photosensitizer, the radioluminescent molecule being suitable for absorbing an X-ray with energy higher than an absorption threshold and for emitting luminescent radiation in the visible domain, and the photosensitizer being suitable for absorbing the luminescent radiation and producing singlet oxygen. The radioluminescent compound is made up of a molecule of lanthanide chloride (LnCl3); the photosensitizer is selected among the following photosensitizers: Al(III)Phthalocyanine, mTHPC, chlorin e6 (Ce6), hypericin, hypocrellin, Nile blue, Oxazine 170, Oxazine 1, Protoporphyrin IX, 7-methoxycoumarin-4-acetic acid, bacteriochlorophyll, and auramin; and the photosensitizer is selected such as to maximize the energy transfer between an X-ray absorbed by the radioluminescent lanthanide 2 and the photosensitizer in order to produce singlet oxygen.

The present invention relates to pharmacological compositions intended for a combined treatment of radiotherapy and photodynamic therapy (PDT). The invention also relates to pharmacological compositions intended for medical imaging, for guiding a combined therapeutical treatment of radiotherapy and photodynamic therapy (PDT).

The PDT is a known treatment that uses the excitation of a photosensitiser by a visible light beam to produce cytotoxic intermediates linked to oxygen, such as the singlet oxygen or free radicals. These cytotoxic intermediates cause the death of the cells and the response of the biological tissue. Indeed, the singlet oxygen easily generates free radicals and its oxidising capacity is far more important than that of the normal oxygen. The PDT has been authorized for the treatment of the age-related macular degeneration (ARMD) or of precancerous conditions such as a superficial cancer of the stomach, the palliative treatment of the head and the neck, and the malignant tumour of skin.

The PDT has little secondary effects as compared to other therapeutical treatments of the cancer, such as surgery, radiotherapy or chemotherapy. However, the PDT is far from being applied in a general manner. The main drawback of the PDT is the limitation of the penetration of the visible light into the biological tissues. Indeed, the application of PDT is limited to the therapy of superficial tissues. The PDT has also certain limitations. In certain cases, the PDT may lead to an extended photosensitization of the body due to a non-specific bio-distribution of the photosensitiser. The main limitation of the PDT is due to the low penetration of the UV-visible light into the tissues. The PDT has a low accessibility to the malignant tumours located in depth.

External light sources, such as lamps or lasers, may be used in a non-invasive manner to reach tumours located in the depth of penetration of the visible light, of the order of 1 cm for the near-infrared wavelengths. As an alternative, the light may be applied in a weakly invasive manner, in interstitial treatments, by bringing an optical fibre inside the tumour through a needle. However, even in this second approach, the distribution of light is not homogeneous and non-identified metastases are left non treated.

There exist regions of the light spectrum where the depth of penetration of the light into the biological tissues is more important. However, the photosensitisers are not absorbing in these regions of the spectrum.

Groups of researchers continue to tent to synthesize new photosensitisers having a better absorption in the optical window of the biological tissues. However, even with a photosensitiser that is absorbent in the near infrared, the depth of penetration of the infrared light into the tissues remains limited to about 1 cm.

To overcome this limit, another technique, called SLPDT for “self-lightning photodynamic therapy”, consists in combining a radioluminescent nanoparticle and a photosensitiser, by attaching one or several molecules of photosensitiser on a radioluminescent nanoparticle. The exposure of the radioluminescent nanoparticle to an X-ray type radiation, as those used in radiotherapy, triggers the emission by radioluminescence of a visible radiation near the photosensitiser, which absorbs the visible radiation and hence liberates singlet oxygen (cf. Radiation Damage in Biomolecular Systems, Chap. 27: Synchrotron radiation and photodynamic therapy, 2012, pp. 445-460, ed. Springer).

Hence, the patent document US2007/0218049_A1 (Wei Chen and Jun Zhang) describes molecules of photosensitiser (such as porphyrins) conjugated by covalent bond to a possibly doped, luminescent nanoparticle, for example made of ZnO or CaF₂. The so-formed nanoparticle is encapsulated to be made hydrophilic and used as an agent for the PDT. After an exposure to an ionizing radiation or to X rays, the nanoparticle emits visible light that activates a photosensitiser. As a consequence, the photosensitiser produces singlet oxygen that is able to increase the mortality of the cancerous cells. In the SLPDT technique, no external source of visible or infrared light is required to activate the photosensitiser agent inside a tumour. According to this document US2007/0218049_A1, to be useful as a photosensitiser carrier, the nanoparticles have to be made hydrophilic and to have a very large active surface. The nanoparticles can penetrate inside the cells due to their nanometric size and may be grafted to a great variety of molecules. However, the nanoparticles show a risk of nanotoxicity. Now, to be used in therapeutical applications, the nanoparticles must be non-toxic, soluble in water and stable in a biological environment. Chen et al. more particularly propose the use of possibly doped radioluminescent nanoparticles of CaF₂, BaFBr, CaPO₄, ZnO and ZnS. However, in case of dopant by a cation (Eu³⁺ or Eu²⁺), Chen indicates that the nanoparticle must be covered with a thin layer of silica to avoid that the cation catches the singlet oxygen emitted by the photosensitiser.

The X rays having a far higher depth of penetration in the biological tissues than the visible rays, the combination of the radiotherapy and PDT techniques supresses the use of an external visible light source and hence makes it possible to extend the applications of the radio-PDT to the therapy of deep tissues. Moreover, the combination of the radiotherapy and the PDT is more efficient than each of both techniques applied alone, which makes it possible to reduce the doses of X rays compared to the radiotherapy used alone.

Moreover, the patent document WO2010/143942_A1 describes the incorporation of magnetic contrast agents in nanoparticles containing a photosensitiser to increase the contrast in magnetic resonance imaging (MRI). The contrast agents are for example obtained by doping with ions Gd³⁺, Fe³⁺ or Mn²⁺. The nanoparticles with magnetic contrast agent make it possible to follow the kinetics of the pharmacological composition in the so-treated organism.

However, it is desirable to further reduce the dose of radiation applied in a combination of X-ray therapies and photodynamic therapy (PDT), to reduce the secondary effects to a minimum while accessing to deep tumours.

There thus exists a need for a system and a method for a photodynamic therapy treatment that is efficient on deep tumour cells which are inaccessible to visible radiation with a reduced dose of radiation.

One of the objects of the invention is to improve the efficiency of cancer treatments by radiotherapy. One of the objects of the invention is to propose new compounds formed of a radioluminescent agent and a photosensitiser making it possible to reach deep tumours without increasing the dose of radiations required for the activation of the photosensitiser, and if possible by reducing the dose of radiations required for the activation of the photosensitiser. Another object of the invention is to improve the energy transfer between a radioluminescent component and a photosensitiser.

Another object of the invention is to propose a device of radiation-induced deep photodynamic therapy of tumours and a method of X-ray-induced photoluminescence.

The present invention has for object to remedy the drawbacks of the prior devices and methods.

The invention relates to a radioluminescent compound for radiotherapy and deep tumours photodynamic therapy (DeepPDT), the radioluminescent compound including a molecular conjugate, the molecular conjugate being consisted of a couple formed of a radioluminescent molecule and a photosensitiser, the radioluminescent molecule being adapted to absorb an X-ray radiation of energy higher than an absorption threshold and to emit a luminescence radiation in the visible domain, and the photosensitiser being adapted to absorb said luminescence radiation and to produce singlet oxygen.

According to the invention, the radioluminescent compound is consisted of a molecule of lanthanide chloride (LnCl₃), in free or aggregated form, said photosensitiser is preferably chosen among the following photosensitisers: Al(III)Phthalocyanine; mTHPC; chlorin e6 (Ce6); hypericin, hypocrellin, Nile blue, Oxazine 170, Oxazine 1, Protoporphyrin IX, 7-Methoxycoumarin-4-acetic acid, Bacteriochlorophyll, Auramin, said molecular conjugate in solution being consisted of a lanthanide chloride associated with the photosensitiser, in a covalent or non-covalent way, and said photosensitiser being selected so as to maximise the energy transfer between an X-ray radiation, absorbed by the radioluminescent lanthanide, and the photosensitiser to produce singlet oxygen.

Under exposure to an X-ray radiation, the molecular conjugate administered to a patient makes it possible to locally deliver singlet oxygen near deep tumours, which are inaccessible by the prior SLPDT techniques. The compound has the advantage not to catch the singlet oxygen emitted.

The compound makes it possible to combine the effects of the radiotherapy and the photodynamic therapy for the treatment of deep tumours.

The molecular conjugate requires no encapsulation and is not necessarily hydrophilic. On the contrary, preferably, the strong hydrophobicity of a photosensitiser or a couple formed of a radioluminescent molecule and a photosensitiser makes it possible to favour the insertion of the radioluminescent compound in the cell membranes or in the lipoproteins of LDL type and hence generally increases the activity of the radioluminescent compound.

According to a particular embodiment, the radioluminescent molecule of lanthanide chloride is chosen among: cerium chloride (CeCl₃), europium chloride (EuCl₃), gadolinium chloride (GdCl₃) and terbium chloride (TbCl₃).

According to a particular embodiment, the molecular conjugate is chosen among the following compounds: cerium chloride (CeCl₃) with Al(III)Phthalocyanine; cerium chloride (CeCl₃) with mTHPC; cerium chloride (CeCl₃) with chlorin e6 (Ce6); europium chloride (EuCL₃) with Hypericin; gadolinium chloride (GdCl₃) with Hypericin; terbium chloride (TbCl₃) with Hypericin; terbium chloride (TbCl₃) with Hypocrellin; europium chloride (EuCl₃) with Nile blue; europium chloride (EuCl₃) with Oxazine 170; europium chloride (EuCl₃) with Oxazine 1; cerium chloride (CeCl₃) with Protoporphyrin IX; cerium chloride (CeCl₃) with the 7-Methoxycoumarin-4-acetic acid; cerium chloride (CeCl₃) with Bacteriochlorophyll; cerium chloride (CeCl₃) with Auramin 0; gadolinium chloride (GdCl₃) with the 7-Methoxycoumarin-4-acetic acid.

Advantageously, the electronic properties of the molecular conjugate are adjusted so as to maximise the energy transfer between the radioluminescent element and the photosensitiser.

Preferably, said compound is in solution in a solvent.

In a particular and advantageous embodiment, the lanthanide chloride is adapted to serve as a contrast agent in medical imaging, such as radiodiagnostic imaging, magnetic resonance imaging (MRI), ultrasonography, visible and near-infrared photodiagnostic imaging.

According to a particular and advantageous aspect, the photosensitiser is adapted to serve as a marker for deep tumour in medical imaging, such as radiodiagnostic imaging, magnetic resonance imaging (MRI), ultrasonography, visible and near-infrared photodiagnostic imaging.

The invention also relates to a device of radiotherapy and deep photodynamic therapy of tumours (DeepPDT), comprising:
an X-ray source, preferably a source of synchrotron radiation, said source being adapted to generate an X-ray radiation of energy higher than an absorption threshold of the lanthanide so as to activate a radioluminescent molecular conjugate;
a molecular conjugate chosen among the following lanthanide chloride-photosensitiser couples: cerium chloride (CeCl₃) with Al(III)Phthalocyanine; cerium chloride (CeCl₃) with mTHPC; cerium chloride (CeCl₃) with chlorin e6 (Ce6); europium chloride (EuCL₃) with Hypericin; gadolinium chloride (GdCl₃) with Hypericin; terbium chloride (TbCl₃) with Hypericin; terbium chloride (TbCl₃) with Hypocrelline; terbium chloride (TbCl₃) with Hypocrellin;
said molecular conjugate being adapted to transmit efficiently an activation by X ray, induce a UV-visible radiation by luminescence and produce singlet oxygen.

The invention also relates to a method of selection of a couple of lanthanide-photosensitiser molecules for deep photodynamic therapy of tumours (DeepPDT), comprising the following steps:
exposing a molecule of lanthanide chloride, preferably chosen among cerium chloride (CeCl₃), europium chloride (EuCl₃), gadolinium chloride (GdCl₃) and terbium chloride (TbCl₃), to a dose of X-ray radiation, preferably of the synchrotron type;
recording the radioluminescence spectrum emitted by said molecule of lanthanide chloride in the UV-visible domain;
recording the UV-visible absorption spectrum of a photosensitiser, preferably chosen among Al(III)Phthalocyanine; mTHPC; chlorine e6 (Ce6); hypericin and hypocrellin;
comparing the emission spectrum of the lanthanide molecule and the absorption spectrum of the photosensitiser molecule;
selecting a couple formed of a molecule of lanthanide chloride and a photosensitiser in which an emission band by radioluminescence of said molecule of lanthanide chloride and an absorption band of said photosensitiser are superimposed;
forming a molecular conjugate by covalent or non-covalent bond in a solvent, the molecular conjugate being consisted of a couple selected at the previous step, formed of a photosensitiser and a radioluminescent element adapted for an efficient transfer of X-ray radiation towards a UV-visible photoemission for the generation of singlet oxygen.

The invention will find a particularly advantageous application in the manufacturing of components for the combined treatment by radiotherapy and by photodynamic therapy.

The present invention also relates to the characteristics that will be revealed in the following description and that will have to be considered in isolation or according to any technically possible combination thereof.

This description, given only by way of non-limiting example, will allow a better understanding of how the invention may be implemented, with reference to the appended drawings, in which:

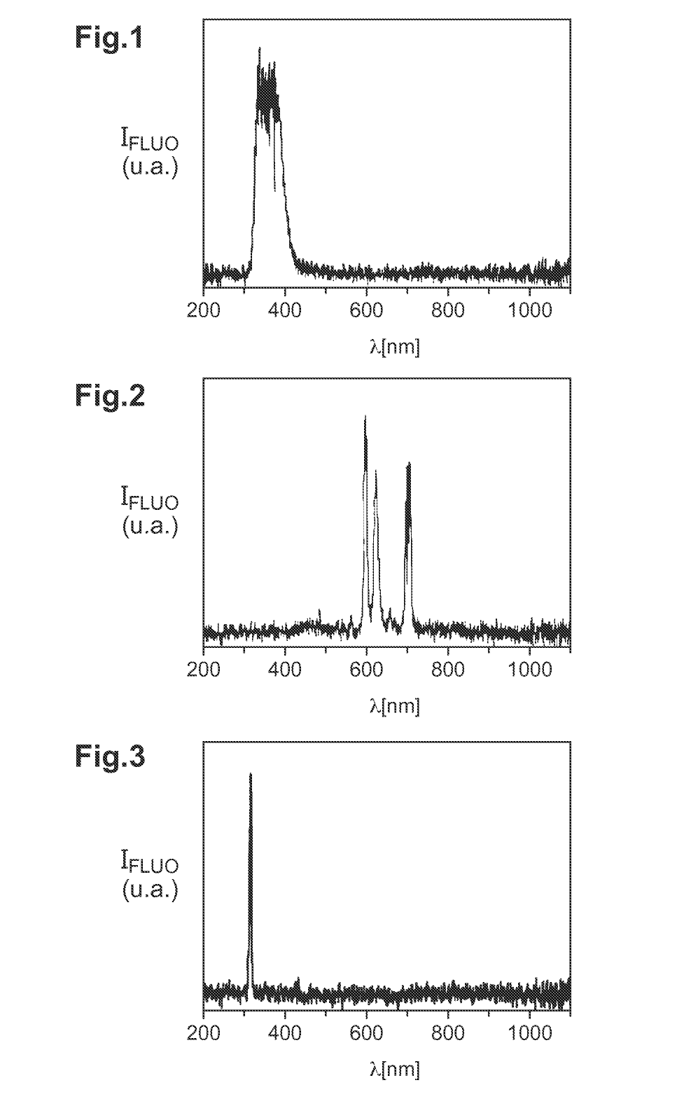

FIG. 1 shows a measurement of spectrum of X-ray-induced luminescence for a molecule of cerium chloride in aqueous medium;

FIG. 2 shows a measurement of spectrum of X-ray-induced luminescence for a molecule of europium chloride in aqueous medium;

FIG. 3 shows a measurement of spectrum of X-ray-induced luminescence for a molecule of gadolinium chloride in aqueous medium;

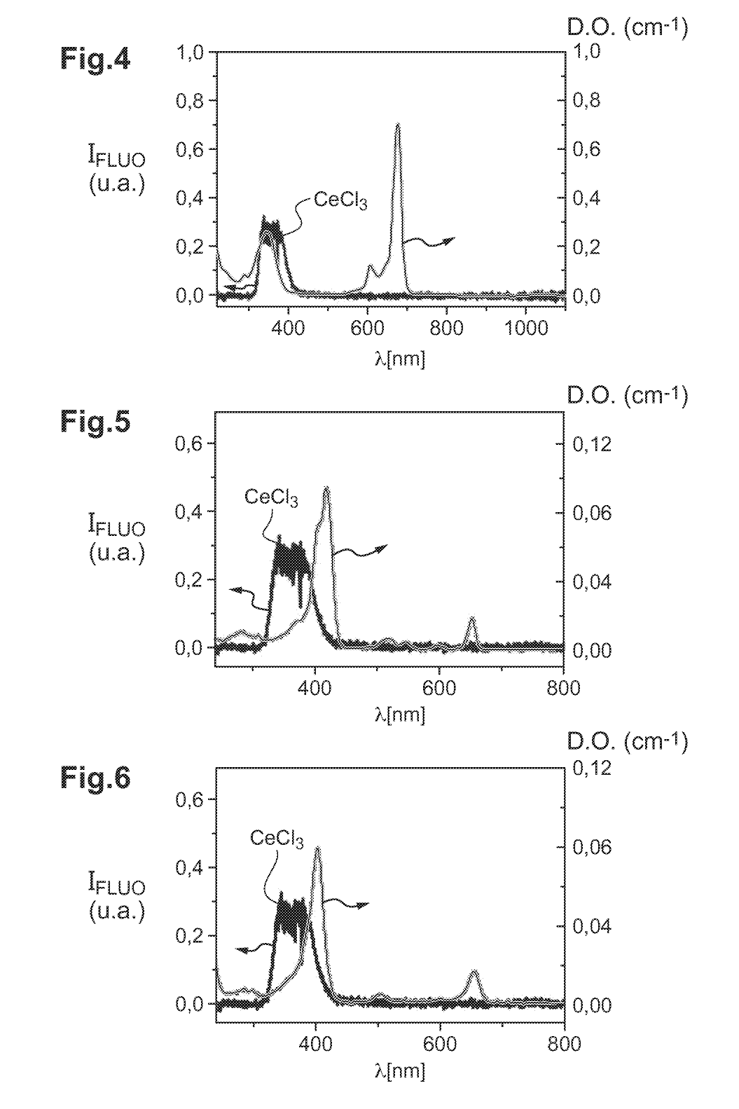

FIG. 4 shows, in superimposition, the radioluminescence spectrum of cerium chloride in aqueous medium and the absorption spectrum of a photosensitiser of aluminium phtalocyanine type in aqueous medium;

FIG. 5 shows, in superimposition, the radioluminescence spectrum of cerium chloride in polyethylene glycol (PEG:EtOH:H₂O) medium and the absorption spectrum of a photosensitiser of the m-tetrahydroxyphenylchlorin (mTHPC) type in PEG:EtOH:H₂O medium;

FIG. 6 shows, in superimposition, the radioluminescence spectrum of cerium chloride in Phosphate Buffer Solution (BPS) and the absorption spectrum of a photosensitiser of aluminium chlorine e6 type in PBS medium;

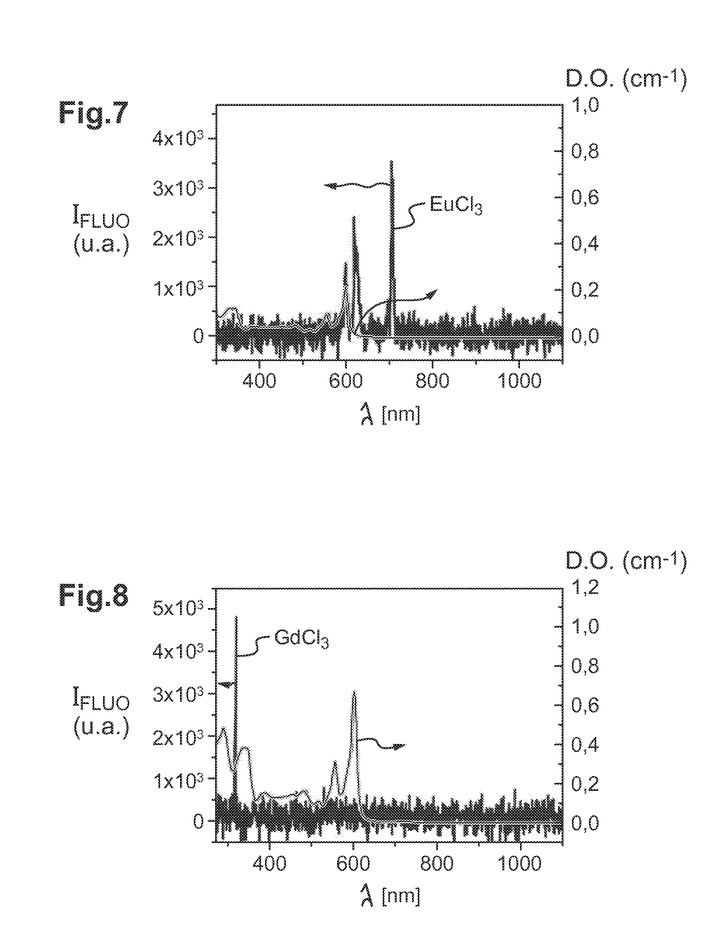

FIG. 7 shows, in superimposition, the radioluminescence spectrum of europium chloride in DMSO medium and the absorption spectrum of a photosensitiser of the Hypericin type in DMSO medium;

FIG. 8 shows, in superimposition, the radioluminescence spectrum of gadolinium chloride in DMSO medium and the absorption spectrum of a photosensitiser of the Hypericin type in DMSO medium.

The invention generally relates to the pharmacological compositions intended for a combined treatment of radiotherapy and photodynamic therapy (PDT) under X-ray exposure.

The invention is linked to the combination of a lanthanide and a photosensitiser to ensure a high energy transfer from the lanthanide to the photosensitiser. The efficiency of the energy transfer is essential for the generation of singlet oxygen, and the key of success for a combined treatment of radiotherapy and PDT.

A selection of a couple formed of a radioluminescent lanthanide element and a photosensitiser is proposed. The lanthanide element and the photosensitiser may be either covalently linked or placed in proximity through a common integration in a vesicule (for example of the SUV: Small Unilamellar Vesicle, GUV: Giant Unilamellar Vesicle, MLV: MultiLamellar Vesicle type), in micelle or in a dendrimer or any other formulation ensuring a sufficient proximity between the two molecules. After an exposure to an X-ray radiation, the lanthanides emit by luminescence a radiation that is generally in the visible spectral domain.

However, the absorption spectrum (or excitation) of a photosensitiser depends not only on its chemical composition, but also on its form and its chemical environment: free photosensitiser in solution, powder photosensitiser or photosensitiser attached to a nanoparticle. The absorption-excitation spectrum of a photosensitiser may also depend on the incident radiation used to excite the photosensitiser. It is hence difficult to provide the absorption-excitation spectrum of a photosensitiser independently of the complete chemical composition and of the final form of the pharmaceutical composition used.

Advantageously, lanthanide ions based on an europium (Eu), cerium (Ce), gadolinium (Gd) or terbium (Tb) element are used.

The photosensitiser is selected among chlorine e6 (Ce6), meta-tetrahydroxyphenylchlorine (mTHPC), aluminium phthalocyanine (Al(III)Phthalocyanine), hypericin and hypocrellin and combinations of these photosensitisers in solution, in micel, liposome, dendrimer or any other formulation ensuring a sufficient proximity between the two molecules.

FIGS. 1-8 show different molecular combinations of lanthanide and/or photosensitiser in various liquid environments, similar results would be obtained in gel.

FIGS. 1-3 show the measurements of spectrum of X-ray-induced luminescence of different molecules based on lanthanides.

More precisely, FIG. 1 shows the X-ray-excited luminescence of molecules of cerium chloride CeCl₃ (concentration c=177 millimolar (mM)) in distilled water. FIG. 2 shows the X-ray-excited luminescence of molecules of gadolinium chloride GdCl₃ (concentration c=199 mM) in distilled water. FIG. 3 shows the X-ray-excited luminescence of molecules of europium chloride EuCl₃ (concentration c=96 mM) in distilled water.

Comparing FIGS. 1 to 3, it is observed that a change of the lanthanide element strongly modifies the spectral position of the X-ray-induced luminescence peak(s).

FIGS. 4-8 show, in superimposition, spectroscopic measurements of radioluminescence of molecules based on lanthanides and spectroscopic measurements of absorption of photosensitisers taken in a same liquid chemical environment.

More precisely, FIG. 4 shows, in superimposition, the spectrum of intensity of X-ray-excited luminescence (ordinate axis, on the left) as a function of the wavelength for a radioluminescent molecule of cerium chloride CeCl₃ (concentration c=177 mM) in an aqueous environment and, respectively, the absorption spectrum (in optical density or D.O. on the right axis) as a function of the wavelength of the photosensitiser Al(III)Phthalocyanine (concentration c=4 micromolar (μM)) in a same aqueous environment. For the acquisition of these spectra, the two molecules are mixed in the same aqueous solvent. The energy of the X-ray radiation is generally comprised between 1 and 20 keV and selected so as to be higher than an absorption threshold of the radioluminescent molecule, so that the radioluminescent molecule absorbs the X-ray radiation of energy and emits a luminescence radiation in the visible domain. The cerium chloride CeCl₃ in aqueous medium shows an emission band between 300 and 400 nm, which is superimposed with an absorption band of the photosensitiser Al(III)Phthalocyanine in aqueous medium around 350 nm. However, the most important absorption peak of the photosensitiser Al(III)Phthalocyanine located between 600 and 700 nm in aqueous medium corresponds to no radioluminescence band of the cerium chloride CeCl₃ in aqueous medium.

Similarly, FIG. 5 shows, in superimposition, the intensity spectrum of X-ray-excited luminescence (ordinate axis, on the left), of energy comprised between 1 and 20 keV, as a function of the wavelength for the same radioluminescent molecule of cerium chloride CeCl₃ (concentration c=177 mM) but located in another environment of polyethylene glycol (PEG:EtOH:H₂O; 3:2:5 v/v) and, respectively, the absorption spectrum (in optical density, right axis) as a function of the wavelength of the photosensitiser mTHPC (concentration c=0.49 μM) in the same aqueous environment of PEG:EtOH:H₂O (3:2:5 v/v). The emission band of cerium chloride CeCl₃ in PEG:EtOH:H₂O medium located between 300-400 nm is superimposed only partially with the most intense absorption band of the photosensitiser mTHPC in PEG:EtOH:H₂O medium around 400 nm.

Similarly, FIG. 6 shows, in superimposition, the intensity of X-ray-excited luminescence (ordinate axis, on the left) as a function of the wavelength for the same radioluminescent molecule of cerium chloride CeCl₃ (concentration c=177 mM) in another environment, herein Phosphate Buffer Solution (PBS) (pH=7.4) and, respectively, the absorption spectrum (in optical density, right axis) as a function of the wavelength of the photosensitiser chlorine e6 (concentration c=0.8 μM) in the same aqueous environment of PBS (pH=7.4). The emission band of the cerium chloride CeCl₃ in PBS medium located between 300-400 nm is superimposed only partially with the most intense absorption band, located around 400 nm, of the photosensitiser chlorine e6 (concentration c=0.8 μM) in a same environment of PBS (pH=7.4).

Let's compare the FIGS. 4-6, where the emission spectrum of a same radioluminescent molecule of cerium chloride CeCl₃ with a same concentration (concentration c=177 mM) is measured in different liquid environments, respectively in aqueous medium (FIG. 4), in PEG:EtOH:H₂O medium (FIG. 5) and in PBS medium (FIG. 6). It is observed that a change of chemical environment of the radioluminescent molecule of cerium chloride CeCl₃ does not modify the spectral position of the luminescence band (300-400 nm) and that the intensity of X-ray-induced luminescence remains similar in the three curves. The luminescence of cerium chloride is observed at the same wavelength in the different chemical environments: distilled water, PBS and PEG:EtOH:H₂O. The photosensitiser the better adapted to cerium chloride seems to be the aluminium phthalocyanine in aqueous medium (illustrated by FIG. 4).

FIG. 7 shows, in superimposition, the intensity of X-ray-excited luminescence (ordinate axis, on the left) as a function of the wavelength for another radioluminescent molecule, herein europium chloride EuCl₃ (concentration c=96 mM) in a dimethylsulfoxide (DMSO) environment and, respectively, the absorption spectrum (in optical density, right axis, as a function of the wavelength on the abscissa axis) of the photosensitiser Hypericin (concentration c=4 μM) in the same DMSO environment. The europium chloride EuCl₃ in DMSO medium shows three bands of emission located around 600 nm; 630 nm and 700 nm, respectively. The emission band of europium chloride EuCl₃ around 600 nm is superimposed with an absorption peak of the photosensitiser Hypericin in DMSO medium around 600 nm.

Comparing the absorption spectrum of europium chloride in aqueous medium in FIG. 2 and, respectively, in DMSO medium in FIG. 7, it is observed that the position of the peaks of absorption does not change, but that the relative intensity of the rays is modified as a function of the chemical environment. In aqueous medium, the absorption peak of europium chloride at 600 nm is the most intense of the three peaks of absorption observed, whereas in DMSO medium, the absorption peak of europium chloride at 700 nm is the most intense of the three peaks of absorption observed.

FIG. 8 shows, in superimposition, the intensity of X-ray-excited luminescence (ordinate axis, on the left) as a function of the wavelength for another radioluminescent molecule, herein gadolinium chloride GdCl₃ (concentration c=199 mM) in a dimethylsulfoxide (DMSO) environment and, respectively, the absorption spectrum (in optical density, right axis, as a function of the wavelength on the abscissa axis) of the photosensitiser Hypericin (concentration c=4 μM) in the same DMSO environment. The gadolinium chloride GdCl₃ in DMSO medium shows a peak of emission located around 320 nm, which is superimposed with a broad absorption band of the photosensitiser Hypericin in DMSO medium from 280 to 600 nm.

FIGS. 1-8 show different molecular combinations of lanthanide and/or photosensitiser in different liquid environments. Similar results would be obtained in gel.

The molecular compound formed of a radioluminescent compound among the lanthanide chlorides and a photosensitiser is selected so as to maximise the energy transfer between the induced radioluminescence energy and the absorption energy of the photosensitiser.

The radioluminescent compound-photosensitiser couple being selected, this couple may then be used in a combined treatment of radiotherapy and PDT.

More precisely, such a process of treatment includes the following steps:
absorption of X rays by a radiosensitiser containing a lanthanide;
emission by the radiosensitiser of a radiation of luminescence or energy transfer;
activation of a photosensitiser triggered by absorption of the luminescence radiation of by non-radiative energy transfer, of the Forster type.

The exposure of the molecular conjugate to the X rays causes an excitation of a luminescent lanthanide ion linked to a photosensitiser. The X-ray-excited lanthanide ion may relax in particular by fluorescence in the visible-UV or also give rise to an energy transfer towards the photosensitiser. The energy transfer between the radiosensitiser and the photosensitiser may be of vibrational type, rotational type, or able to induce a change of electronic level in a molecule. The photosensitiser may also absorb the fluorescence emitted by the lanthanide. The molecular conjugate based on a lanthanide-photosensitiser couple is selected so that an efficient energy transfer is performed between the lanthanide and the photosensitiser.

When a lanthanide-photosensitiser conjugate that is targeted towards a tumour is stimulated by X rays, in particular during a ratiotherapy, the lanthanide generates UV-visible light liable to activate the photosensitiser. Hence, the X-ray exposure and the PDT are combined and occur simultaneously and at the same place. The tumour destruction is hence more efficient. The limit of depth of penetration into the tissues is far higher for the X rays than for an UV-visible radiation. The DeepPDT hence makes it possible to exceed the main limitation of the PDT. Hence, the DeepPDT may be used for the treatment of deep tumours.

According to this method, the conventional radiotherapy is completed by the DeepPDT, which makes it possible to reduce the X-ray doses, making the radiotherapy more efficient and more safe.

Advantageously, other technical effects of this molecular conjugate are that:
the lanthanide chloride may serve as a contrast agent for medical imaging, and/or
the photosensitiser may serve as a marker for a deep tumour.

Other examples of molecular conjugates formed of a radioluminescent lanthanide chloride and a photosensitiser are:
europium chloride (EuCl₃) with Oxazine 170; europium chloride (EuCl₃) with Oxazine 1; europium chloride (EuCl₃) with a mixture of Nile blue and/or Oxazine 170 and/or Oxazine 1;
cerium chloride (CeCl₃) with Protoporphyrin IX; cerium chloride (CeCl₃) with 7-Methoxycoumarin-4-acetic acid; cerium chloride (CeCl₃) with Bacteriochlorophyll; cerium chloride (CeCl₃) with Auramin 0; cerium chloride (CeCl₃) with a mixture of Protoporphyrin IX and/or 7-Methoxycoumarin-4-acetic acid and/or Bacteriochlorophyll and/or Auramin 0;
gadolinium chloride (GdCl₃) with 7-Methoxycoumarin-4-acetic acid.

The couple selected allows an efficient energy transfer between the absorption of X-ray radiation by the radioluminescent lanthanide and the absorption of visible luminescent radiation by the photosensitiser. The transfer efficiency is measured by the measurement of the intensity of fluorescence of the acceptor as a function of the excitation of the energy donor.

The use of a selected lanthanide-photosensitiser couple also makes it possible to increase the contrast in magnetic imaging, for example in magnetic resonance imaging (RMI). This property may be used to make the image of the site to be treated before applying the therapy.

1-9. (canceled)

10. A radioluminescent compound for radiotherapy and deep photodynamic therapy of tumours (DeepPDT), the radioluminescent compound including a molecular conjugate, the molecular conjugate comprising a couple formed of a radioluminescent molecule and a photosensitiser, the radioluminescent molecule being adapted to absorb an X-ray radiation of energy higher than an absorption threshold and to emit a luminescence radiation in the visible domain, and the photosensitiser being adapted to absorb said luminescence radiation and to produce singlet oxygen,
wherein:
the radioluminescent compound comprises a molecule of lanthanide chloride (LnCl₃), in free or aggregated form, said photosensitiser is preferably chosen among the following photosensitisers: Al(III)Phthalocyanine; mTHPC; chlorin e6 (Ce6); hypericin, hypocrellin, Nile blue, Oxazine 170, Oxazine 1, Protoporphyrin IX, 7-Methoxycoumarin-4-acetic acid, Bacteriochlorophyll, Auramin, said molecular conjugate in solution comprising a lanthanide chloride associated with the photosensitiser, in a covalent or non-covalent way, and said photosensitiser being selected so as to maximise the energy transfer between an X-ray radiation, absorbed by the radioluminescent lanthanide, and the photosensitiser to produce singlet oxygen.

11. The radioluminescent compound for radiotherapy and deep photodynamic therapy of tumours (DeepPDT) according to claim 10, wherein the radioluminescent molecule of lanthanide chloride is chosen among: cerium chloride (CeCl₃), europium chloride (EuCl₃), gadolinium chloride (GdCl₃) and terbium chloride (TbCl₃).

12. The radioluminescent compound for radiotherapy and deep photodynamic therapy of tumours (DeepPDT) according to claim 11, wherein the molecular conjugate is chosen among the following compounds: cerium chloride (CeCl₃) with Al(III)Phthalocyanine; cerium chloride (CeCl₃) with mTHPC; cerium chloride (CeCl₃) with chlorin e6 (Ce6); europium chloride (EuCL₃) with Hypericin; gadolinium chloride (GdCl₃) with Hypericin; terbium chloride (TbCl₃) with Hypericin; terbium chloride (TbCl₃) with Hypocrellin; europium chloride (EuCl₃) with Nile blue; europium chloride (EuCl₃) with Oxazine 170; europium chloride (EuCl₃) with Oxazine 1; cerium chloride (CeCl₃) with Protoporphyrin IX; cerium chloride (CeCl₃) with the 7-Methoxycoumarin-4-acetic acid; cerium chloride (CeCl₃) with Bacteriochlorophyll; cerium chloride (CeCl₃) with Auramin O; gadolinium chloride (GdCl₃) with the 7-Methoxycoumarin-4-acetic acid.

13. The radioluminescent compound for radiotherapy and deep photodynamic therapy of tumours (DeepPDT) according to claim 10, wherein the electronic properties of the molecular conjugate are adjusted so as to maximise the energy transfer between the radioluminescent element and the photosensitiser.

14. The radioluminescent compound for radiotherapy and deep photodynamic therapy of tumours (DeepPDT) according to claim 10, wherein said compound is in solution in a solvent.

15. The radioluminescent compound for radiotherapy and deep photodynamic therapy of tumours (DeepPDT) according to claim 10, wherein the lanthanide chloride is adapted to serve as a contrast agent in medical imaging, such as radiodiagnostic imaging, magnetic resonance imaging (MRI), ultrasonography, visible and near-infrared photodiagnostic imaging.

16. The radioluminescent compound for radiotherapy and deep photodynamic therapy of tumours (DeepPDT) according to claim 10, wherein the photosensitiser is adapted to serve as a marker for deep tumour in medical imaging, such as radiodiagnostic imaging, magnetic resonance imaging (MRI), ultrasonography, visible and near-infrared photodiagnostic imaging.

17. A device of radiotherapy and deep photodynamic therapy of tumours (DeepPDT), comprising:
an X-ray source, preferably a source of synchrotron radiation, said source being adapted to generate an X-ray radiation of energy higher than an absorption threshold of the lanthanide so as to activate a radioluminescent molecular conjugate;
a molecular conjugate chosen among the following lanthanide chloride-photosensitiser couples: cerium chloride (CeCl₃) with Al(III)Phthalocyanine; cerium chloride (CeCl₃) with mTHPC; cerium chloride (CeCl₃) with chlorin e6 (Ce6); europium chloride (EuCL₃) with Hypericin; gadolinium chloride (GdCl₃) with Hypericin; terbium chloride (TbCl₃) with Hypericin; terbium chloride (TbCl₃) with Hypocrelline; terbium chloride (TbCl₃) with Hypocrellin;
said molecular conjugate being adapted to transmit efficiently an activation by X ray, induce a UV-visible radiation by luminescence and produce singlet oxygen.

18. A method of selection of a lanthanide-photosensitiser molecules couple for deep photodynamic therapy of tumours (DeepPDT), comprising the following steps:
exposing a molecule of lanthanide chloride, preferably chosen among cerium chloride (CeCl₃), europium chloride (EuCl₃), gadolinium chloride (GdCl₃) and terbium chloride (TbCl₃), to a dose of X-ray radiation, preferably of the synchrotron type;
recording the radioluminescence spectrum emitted by said molecule of lanthanide chloride in the UV-visible domain;
recording the UV-visible absorption spectrum of a photosensitiser, preferably chosen among Al(III)Phthalocyanine; mTHPC; chlorine e6 (Ce6); hypericin and hypocrellin;
comparing the emission spectrum of the lanthanide molecule and the absorption spectrum of the photosensitiser molecule;
selecting a couple formed of a molecule of lanthanide chloride and a photosensitiser in which an emission band by radioluminescence of said molecule of lanthanide chloride and an absorption band of said photosensitiser are superimposed;
forming a molecular conjugate by covalent or non-covalent bond in a solvent, the molecular conjugate comprising a couple selected at the previous step, formed of a photosensitiser and a radioluminescent element adapted for an efficient transfer of X-ray radiation towards a UV-visible photoemission for the generation of singlet oxygen.

19. The radioluminescent compound for radiotherapy and deep photodynamic therapy of tumours (DeepPDT) according to claim 11, wherein the electronic properties of the molecular conjugate are adjusted so as to maximise the energy transfer between the radioluminescent element and the photosensitiser.

20. The radioluminescent compound for radiotherapy and deep photodynamic therapy of tumours (DeepPDT) according to claim 12, wherein the electronic properties of the molecular conjugate are adjusted so as to maximise the energy transfer between the radioluminescent element and the photosensitiser.

21. The radioluminescent compound for radiotherapy and deep photodynamic therapy of tumours (DeepPDT) according to claim 11, wherein said compound is in solution in a solvent.

22. The radioluminescent compound for radiotherapy and deep photodynamic therapy of tumours (DeepPDT) according to claim 12, wherein said compound is in solution in a solvent.

23. The radioluminescent compound for radiotherapy and deep photodynamic therapy of tumours (DeepPDT) according to claim 13, wherein said compound is in solution in a solvent.

24. The radioluminescent compound for radiotherapy and deep photodynamic therapy of tumours (DeepPDT) according to claim 11, wherein the lanthanide chloride is adapted to serve as a contrast agent in medical imaging, such as radiodiagnostic imaging, magnetic resonance imaging (MRI), ultrasonography, visible and near-infrared photodiagnostic imaging.

25. The radioluminescent compound for radiotherapy and deep photodynamic therapy of tumours (DeepPDT) according to claim 12, wherein the lanthanide chloride is adapted to serve as a contrast agent in medical imaging, such as radiodiagnostic imaging, magnetic resonance imaging (MRI), ultrasonography, visible and near-infrared photodiagnostic imaging.

26. The radioluminescent compound for radiotherapy and deep photodynamic therapy of tumours (DeepPDT) according to claim 14, wherein the lanthanide chloride is adapted to serve as a contrast agent in medical imaging, such as radiodiagnostic imaging, magnetic resonance imaging (MRI), ultrasonography, visible and near-infrared photodiagnostic imaging.

27. The radioluminescent compound for radiotherapy and deep photodynamic therapy of tumours (DeepPDT) according to claim 11, wherein the photosensitiser is adapted to serve as a marker for deep tumour in medical imaging, such as radiodiagnostic imaging, magnetic resonance imaging (MRI), ultrasonography, visible and near-infrared photodiagnostic imaging.

28. The radioluminescent compound for radiotherapy and deep photodynamic therapy of tumours (DeepPDT) according to claim 12, wherein the photosensitiser is adapted to serve as a marker for deep tumour in medical imaging, such as radiodiagnostic imaging, magnetic resonance imaging (MRI), ultrasonography, visible and near-infrared photodiagnostic imaging.

29. The radioluminescent compound for radiotherapy and deep photodynamic therapy of tumours (DeepPDT) according to claim 14, wherein the photosensitiser is adapted to serve as a marker for deep tumour in medical imaging, such as radiodiagnostic imaging, magnetic resonance imaging (MRI), ultrasonography, visible and near-infrared photodiagnostic imaging.
